(19) United States
(12) Patent Application Publication
(10) Pub. No.: US 2004/0044362 A1
(43) Pub. Date: Mar. 4, 2004

(54) Acupressure device for relieving motion sickness and method therefor

(76) Inventor: Emilie C. Klassen, Mesa, AZ (US)

Correspondence Address: WEISS & MOY PC, 4204 NORTH BROWN AVENUE, SCOTTSDALE, AZ, 85251, US

(21) Appl. No.: 10/235,134

(22) Filed: Sep. 3, 2002

(51) Int. Cl. A61B 17/00

(52) U.S. Cl. 606/201

(57) ABSTRACT

A clasp for securing a piece of jewelry around a wrist of a wearer and for relieving symptoms of motion sickness has a base member. A protrusion is formed on a bottom surface of the base member. The protrusion will apply pressure to a wrist area of a wear to relieve symptoms of motion sickness. A locking arm is hingely coupled to the base member. The locking arm is used for securing the clasp around the wrist of the wearer.

### BACKGROUND OF THE INVENTION

1. Field of the Invention

This invention relates generally to jewelry and, more specifically, to a clasp for securing a piece of jewelry to a user's wrist wherein the clasp has a protrusion for applying pressure to the wrist of the wearer in order to relieve the wearer from the symptoms of motion sickness.

2. Description of the Prior Art

When traveling in an automobile, train, airplane, ship, or the like, many people tend to suffer from motion sickness. Sometimes the motion sickness is so severe that the person traveling must stop the trip. This is extremely difficult if a person is flying or aboard a ship which is not scheduled to stop for some time.

Many people take anti-nausea medication when feeling the effects of motion sickness. Although there are many effective anti-nausea medications available today, there are also drawbacks associated with the use of chemical compositions. For example, many people are allergic to some chemical medications and therefore cannot use this form of treatment to alleviate nausea. Furthermore, many people would prefer not to use medication as a means to alleviate motion sickness. Consequently, the need to relieve nausea by means other than chemical medications has developed.

The use of acupuncture and acupressure has effectively been in use for many centuries in other countries. Acupuncture and acupressure are traditional Chinese therapeutic techniques involving the stimulation of certain nerves and nerve junctions by puncturing the skin with fine needles or manual massage and pressure of those same nerve junctions to produce a certain physiological effect. The exact means by which this is accomplished is unclear, but the results are conclusive.

Therefore, a need existed to provide an acupressure device for relieving the symptoms of motion sickness. The acupressure device should be unobtrusive so as to be worn by the user but not noticeable to others around.

### SUMMARY OF THE INVENTION

In accordance with one embodiment of the present invention, it is an object of the present invention to provide an acupressure device for relieving the symptoms of motion sickness.

It is another object of the present invention to provide an acupressure device for relieving the symptoms of motion sickness wherein the acupressure device is unobtrusive so as to be worn by the user but not noticeable to others around.

### BRIEF DESCRIPTION OF THE EMBODIMENTS

In accordance with one embodiment of the present invention, a clasp for securing a piece of jewelry around a wrist of a wearer and for relieving symptoms of motion sickness is disclosed. The clasp has a base member. A protrusion is formed on a bottom surface of the base member. The protrusion will apply pressure to a wrist area of a wear to relieve symptoms of motion sickness. A locking arm is hingely coupled to the base member. The locking arm is used for securing the clasp around the wrist of the wearer.

In accordance with another embodiment of the present invention, a clasp for securing a piece of jewelry around a wrist of a wearer and for relieving symptoms of motion sickness is disclosed. The clasp has a base member. A slot is formed on a proximate end of the base member for securing a first end of the piece of jewelry to the clasp. A protrusion is formed on a bottom surface of the base member. The protrusion will apply pressure to a wrist area of a wear to relieve symptoms of motion sickness. A locking arm is hingely coupled to the base member for securing the clasp around the wrist of the wearer. A locking pin is used for hingely coupling the base member to the locking arm and for coupling a second end of the jewelry to the clasp.

The foregoing and other objects, features, and advantages of the invention will be apparent from the following, more particular, description of the preferred embodiments of the invention, as illustrated in the accompanying drawing.

### BRIEF DESCRIPTION OF THE DRAWINGS

The novel features believed characteristic of the invention are set forth in the appended claims. The invention itself, as well as a preferred mode of use, and advantages thereof, will best be understood by reference to the following detailed description of illustrated embodiments when read in conjunction with the accompanying drawings.

FIG. 1 is an elevated perspective view of a clasp which is used to relieve the symptoms of motion sickness which is placed on the wrist of a user.

FIG. 2 is an elevated perspective view of the clasp which relieves the symptoms of motion sickness of the present invention.

FIG. 3 is a cross-section view of the clasp of the present invention taken along lines 3-3 of FIG. 2.

FIG. 4 is a side view of the clasp with the arm of the clasp in an open and unlocked position.

FIG. 5 is a bottom view of the clasp of the present invention.

FIG. 6 is an exploded view of the clasp of the present invention.

### DETAILED DESCRIPTION OF THE PREFERRED EMBODIMENT

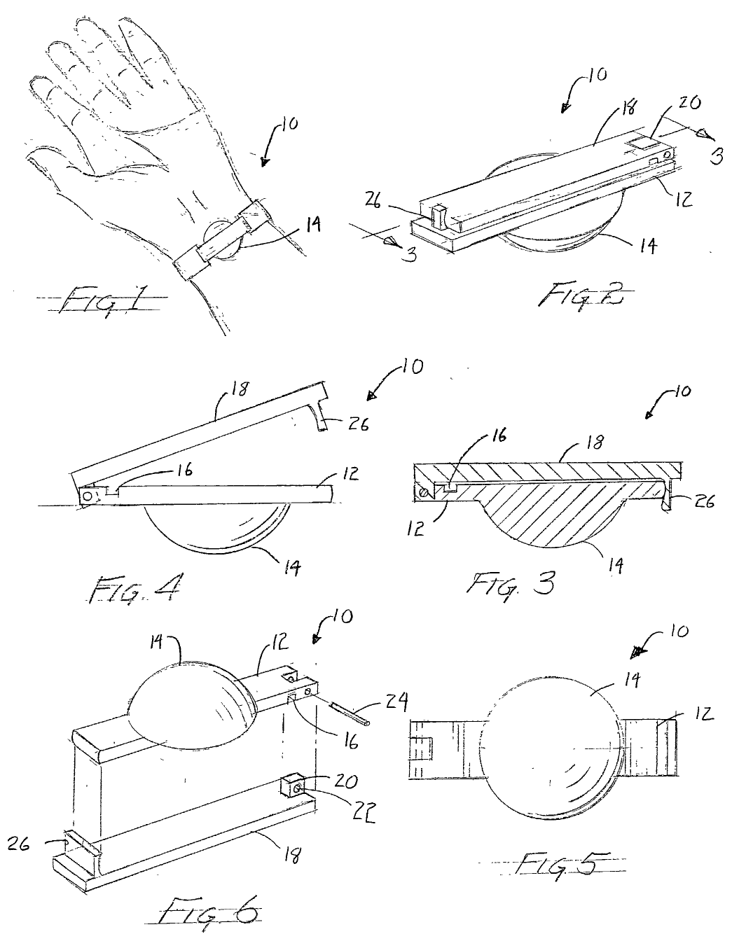

Referring to FIGS. 1-6 wherein like numerals and symbols represent like elements a clasp 10 is shown. The clasp 10 is used to secure two ends of a piece of jewelry around the wrist of a wearer. For example, the clasp 10 may be used to secure a bracelet, a watch, or the like around the wrist of the wearer. What is unique about the clasp 10 is that the clasp 10 will apply a slight amount of pressure to the wrist of the wearer in order to relieve the symptoms of motion sickness.

The clasp 10 may come in many different sizes and shapes. The clasp 10 may further be made out of many different types of materials. In general, the clasp 10 will be made from a material similar to that which the jewelry in which the clasp 10 will be attached is made from.

The clasp 10 has a base member 12. The base member 12 may come in any size or shape. In the embodiment depicted in the Figures, the base member 12 is rectangular in shape. However, this is just given as an example and should not be seen as to limit the scope of the present invention.

On the bottom section of the base member 12 is a protrusion 14. The protrusion 14 is used to apply pressure to the percardium six (P6) acupressure point on the wrist of the wearer. The percardium six (P6) is the acupressure point for relieving nausea. In the embodiment depicted in the Figures, the protrusion 14 is semispherical in shape. However, this should not be seen as to limit the scope of the present invention. Other shapes may be used as long as the protrusion applies pressure to the percardium six (P6) acupressure point on the wrist of the wearer.

On the top surface of base member 12 is a slot 16. The slot 16 is generally positioned on the proximal end of the base member 12. However, this should not be seen as to limit the scope of the present invention. The slot 16 may be positioned anywhere on the top surface of base member 12. The slot 16 is used to secure one end of a piece of jewelry within the clasp 10 so as to secured the piece of jewelry around the wrist of the wearer.

Hingely coupled to the base member 12 is a locking arm 18. The locking arm 18 will lock the clasp 10 in place around the wrist of the wearer securing the piece of jewelry also around the wrist of the wearer. In the embodiment depicted in the Figures, the locking arm 18 has a small step 20 coupled to the proximate end of the locking arm 18. The step 20 has an opening 22 formed there through. A locking pin 24 is then placed through an opening in the base member 12 and through the opening 22 hingely coupling the locking arm 18 to the base member.

A tab member 26 extends down from the locking arm 18. The tab member 26 extends from the distal end of the locking arm 18. The tab member 26 will secure the locking arm 18 in a closed position over the base member 12 securing the clasp 10 around the wrist of the wearer.

Jewelry is generally coupled to the clasp 10. In accordance with one embodiment of the present invention, one end of the jewelry will have a channeling. When the channeling is properly aligned, one may insert the locking pin 24 through the channeling, the opening in the base member 12, through the opening 22 in the step 20 and then back through the base member 12 and the channeling in the jewelry. This will secure one end of the jewelry to the clasp 10. The other end of the jewelry will generally have a ring member. The ring member is positioned in the slot 16. The locking arm 18 is then closed securing the other end of the jewelry in the clasp 10. When properly secured around the wrist of the wearer, the protrusion 14 will press into the wrist of the wearer. The protrusion 14 will apply pressure to the percardium six (P6) acupressure point on the wrist of the wearer. The percardium six (P6) is the acupressure point for relieving nausea.

While the invention has been particularly shown and described with reference to preferred embodiments thereof, it will be understood by those skilled in the art that the foregoing and other changes in form and details may be made therein without departing from the spirit and scope of the invention.

What is claimed is:

1. A clasp for securing a piece of jewelry around a wrist of a wearer and for relieving symptoms of motion sickness comprising:
a base member;
a protrusion formed on a bottom surface of the base member wherein the protrusion will apply pressure to a wrist area of a wear to relieve symptoms of motion sickness; and
a locking arm hingely coupled to the base member for securing the clasp around the wrist of the wearer.

2. A clasp for securing a piece of jewelry around a wrist of a wearer and for relieving symptoms of motion sickness in accordance with claim 1 further comprising a slot formed on a proximate end of the base member for securing one end of the jewelry to the clasp.

3. A clasp for securing a piece of jewelry around a wrist of a wearer and for relieving symptoms of motion sickness in accordance with claim 1 wherein the protrusion is semispherical in shape.

4. A clasp for securing a piece of jewelry around a wrist of a wearer and for relieving symptoms of motion sickness in accordance with claim 1 wherein the protrusion applies pressure to the percardium six (P6) acupressure point on the wrist of the wearer to relieve motion sickness when the clasp is placed around the wrist of the wearer.

5. A clasp for securing a piece of jewelry around a wrist of a wearer and for relieving symptoms of motion sickness in accordance with claim 1 further comprising a locking pin for hingely coupling the base member to the locking arm and to coupled one end of the jewelry to the clasp.

6. A clasp for securing a piece of jewelry around a wrist of a wearer and for relieving symptoms of motion sickness in accordance with claim 1 wherein the locking arm has a tab member extending from a distal end of the locking arm wherein the tab member will engage a distal end of the base member thereby locking the clasp around the wrist of the wearer.

7. A clasp for securing a piece of jewelry around a wrist of a wearer and for relieving symptoms of motion sickness comprising:
a base member;
a slot formed on a proximate end of the base member for securing a first end of the piece of jewelry to the clasp;
a protrusion formed on a bottom surface of the base member wherein the protrusion will apply pressure to a wrist area of a wear to relieve symptoms of motion sickness;
a locking arm hingely coupled to the base member for securing the clasp around the wrist of the wearer; and
a locking pin for hingely coupling the base member to the locking arm and for coupling a second end of the jewelry to the clasp.

8. A clasp for securing a piece of jewelry around a wrist of a wearer and for relieving symptoms of motion sickness in accordance with claim 1 wherein the protrusion is semispherical in shape.

9. A clasp for securing a piece of jewelry around a wrist of a wearer and for relieving symptoms of motion sickness in accordance with claim 1 wherein the protrusion applies pressure to the percardium six (P6) acupressure point on the wrist of the wearer to relieve motion sickness when the clasp is placed around the wrist of the wearer.

10. A clasp for securing a piece of jewelry around a wrist of a wearer and for relieving symptoms of motion sickness in accordance with claim 1 wherein the locking arm has a tab member extending from a distal end of the locking arm wherein the tab member will engage a distal end of the base member thereby locking the clasp around the wrist of the wearer.
